(19) United States
(12) Patent Application Publication
(10) Pub. No.: US 2019/0142287 A1
(43) Pub. Date: May 16, 2019

(54) Optical Heart Rate Sensor

(71) Applicant: OSRAM OPTO SEMICONDUCTORS GMBH, Regensburg (DE)

(72) Inventors: Tim Böscke, Regensburg (DE); Tilman Rügheimer, Regensburg (DE)

(21) Appl. No.: 15/571,781

(22) PCT Filed: May 4, 2016
(86) PCT No.: PCT/EP2016/060037

§ 371 Date: Nov. 3, 2017

(30) Foreign Application Priority Data
May 5, 2015 (DE) 10 2015 106 995.4

(51) Int. Cl. A61B 5/024

(52) U.S. Cl. CPC A61B 5/02427; A61B 2562/0233

(57) ABSTRACT

An optical heart rate sensor is disclosed. In an embodiment the optical heart rate sensor includes at least one light source and at least one photodetector, wherein the light source comprises a blue light-emitting diode with a conversion phosphor, and wherein the conversion phosphor converts the blue light into a green-yellow light.

This patent application is a national phase filing under section 371 of PCT/EP2016/060037, filed May 4, 2016, which claims the priority of German patent application 10 2015 106 995.4, filed May 5, 2015, each of which is incorporated herein by reference in its entirety.

### TECHNICAL FIELD

The invention relates to an optical heart rate sensor.

### BACKGROUND

Optical heart rate sensors can be realized by virtue of radiating the light from a light-emitting diode onto the skin. Here, the light is scattered by the tissue under the skin and the intensity of the scattered light can be measured by a photodetector. Moreover, some of the radiated light is absorbed by the hemoglobin molecules in the blood. Driven by the heart, the blood is pumped through the arteries, with the amount of blood in an artery not being constant but pulsing at the same frequency as the heart rate. As a result, the amount of blood in the artery varies with the heart rate, just like there is variation in the amount of available hemoglobin. More or less of the light from the light emitting diode is absorbed by the hemoglobin depending on whether more or less hemoglobin is present in the artery. As a result, the intensity of the scattered light likewise varies with the heart rate. This varying intensity can be detected by the photodetector. As a result, the heart rate can be deduced from the variation in the photocurrent of the photodetector. Such an optical heart rate sensor is known from DE 10 2008 022 920 B4. This optical heart rate sensor uses a light-emitting diode with a wavelength of 590 nm. The oxyhemoglobin that is particularly well suited to the optical determination of the heart rate has an absorption maximum in the region of 570 nm. Oxyhemoglobin is the hemoglobin containing oxygen which occurs in the arteries in particular.

### SUMMARY OF THE INVENTION

Embodiments provide an improved optical heart rate sensor comprising a light source that is matched to the absorption properties of hemoglobin.

In various embodiments an optical heart rate sensor comprises at least one light source and at least one photodetector, wherein a blue light-emitting diode with a conversion phosphor is used as a light source. This conversion phosphor is embodied in such a way that it at least partly converts the blue light from the light-emitting diode into green-yellow light, with the green-yellow light having a wavelength of between 540 and 585 nm. Hemoglobin has an absorption maximum for green-yellow light in the region of 570 nm. As a result, the use of green-yellow light for an optical heart rate sensor is advantageous. However, conventional green-yellow light-emitting diodes do not offer enough power for use in an optical heart rate sensor. Therefore, it is advantageous to convert light from a light-emitting diode with a shorter wavelength into green-yellow light by means of a conversion phosphor. Particularly the combination of a blue light-emitting diode with a conversion phosphor that completely converts the blue light into green-yellow light is suitable as a light source for an optical heart rate sensor.

In an embodiment, at least 25%, preferably at least 40%, particularly preferably at least 60% of the converted light has a wavelength of between 540 nm and 585 nm. The absorption maximum of the hemoglobin is at 570 nm. As a result, green-yellow light with a wavelength of between 540 nm and 585 nm hits this absorption maximum particularly well. At most 25%, preferably at most 15%, particularly preferably 8% of the converted light has a wavelength longer than 600 nm. There is little absorption by hemoglobin molecules in the wavelength range above 600 nm, leading to a reduction in the pulsing component in the measurement signal. It is for this reason that the component of the light in the wavelength range above 600 nm should be as small as possible so that the signal-to-noise ratio becomes as large as possible. As a result, the aforementioned converted light is particularly well suitable for a light source for an optical heart rate sensor.

In an embodiment, the blue light-emitting diode is an indium gallium nitride LED (InGaN LED). InGaN LEDs have a high output power of the blue light. By combining a blue InGaN LED with a conversion layer, it is possible to provide a green-yellow light with a higher intensity than compared with a conventional green-yellow light-emitting diode. As a result, exploiting the absorption maximum of the hemoglobin at 570 nm is facilitated. This absorption maximum cannot be exploited using conventional green-yellow light-emitting diodes since the output power of the green-yellow light-emitting diode would not be high enough.

In an embodiment, the blue light-emitting diode has a wavelength of between 400 nm and 450 nm. These wavelengths are typical wavelengths for blue InGaN LEDs.

In an embodiment, the InGaN LED has an overall efficiency of at least 40%. With an overall efficiency of at least 40%, a light yield of the blue light-emitting diode that is ideal for the application in an optical heart rate sensor is achieved.

In an embodiment, the conversion phosphor comprises cerium-doped lutetium aluminum garnet (LuAG). Lutetium aluminum garnet is a colorless material that is transparent in the ultraviolet and blue spectral range. As a result of doping with cerium, conversion phosphor that absorbs blue light and emits green-yellow light arises. As a result, the blue light is converted into green-yellow light. Compared with other phosphors, such as, e.g., cerium-doped ytterbium aluminum garnet, cerium-doped lutetium aluminum garnet has a wavelength that covers the green-yellow spectral range, and in particular the absorption maximum of the hemoglobin, in an improved manner. The light converted by means of cerium-doped lutetium aluminum garnet has, in particular, only a small component of converted light with a wavelength that is longer than 600 nm. This is advantageous as a greater component of the light is scattered by the tissue at a wavelength longer than 600 nm, while there is only little absorption of the light in the hemoglobin molecules. As a result, much scattered light reaches the photodetector while there is only little absorption by the pulsating arterial blood. As a result, the signal-to-noise ratio drops.

In an embodiment, the cerium concentration in the lutetium aluminum garnet is 1%. A 1% concentration of cerium in lutetium aluminum garnet covers the wavelength range from 500 nm to 570 nm very well and is therefore particularly well suited to an optical heart rate sensor. Moreover, what a cerium concentration of 1% in the lutetium aluminum garnet achieves is that little light with a wavelength of longer than 600 nm arises during the conversion.

In an embodiment, the conversion phosphor, which consists of cerium-doped lutetium aluminum garnet, has been introduced in powder form in another material. Here, the grain size of the powder is in the micrometer range. Here, the other material can be epoxy resin, silicone, a plastic or a ceramic. As a result, it is possible to produce a conversion element that is relatively cost-effective. By introducing the cerium-doped lutetium aluminum garnet in powder form, it is not necessary to produce perfect lutetium aluminum garnet crystals. As a result, the production process of the cerium-doped lutetium aluminum garnet is significantly simplified, allowing costs to be saved.

In an embodiment, the conversion phosphor comprises quantum dots. Quantum dots are nanoscale material structures, in which charge carriers (electrons and/or holes) are restricted in terms of their movement in all three spatial directions such that the energy thereof can no longer assume continuous values, but can only still have discrete values. Thus, quantum dots have a similar behavior to atoms within a solid. It is for this reason that quantum dots are likewise well suited to the conversion of blue light into green-yellow light with a wavelength range of 540 nm to 585 nm. Quantum dots have a relatively narrow band emission spectrum. Green-yellow light with a narrow band wavelength distribution in the region of the absorption maximum of the hemoglobin is produced as a result of selecting quantum dots as a conversion material. Here, the quantum dots have a diameter of between 2 and 6 nm.

In an embodiment, the quantum dots comprise mercury sulfide, lead sulfide, cadmium sulfide, cadmium selenide, indium arsenide or indium phosphide. The conversion wavelength of 570 nm can be obtained with quantum dots made of the aforementioned materials. The converted light has a distribution of ±15 nm about this wavelength of 570 nm. Expressed differently, this means that a green-yellow converted light with a wavelength of between 555 nm and 585 nm is produced with quantum dots having a diameter of between 2 and 6 nm. This light is well suited for use in an optical heart rate sensor.

In an embodiment, provision is made for the light source or the photodetector to comprise a filter which is transmissive for some of the converted green-yellow light. As a result of this, it is possible to filter out components of the converted light that do not lie in the ideal spectral range. As a result, these wavelengths no longer impinge on the photodetector as scattered light, as a result of which a cleaner signal is produced. In particular, this allows a component of the green-yellow light in the photodetector to be increased, as a result of which an improved signal-to-noise ratio simplifies the determination of the heart rate.

In an embodiment, the light source or the photodetector may comprise a filter which is transmissive for the wavelength range of 540 nm to 585 nm. In this case too, what the filter achieves is that bothersome scattered light in wavelength regions that are not relevant for the absorption of the light in the hemoglobin are filtered out. As a result, an improved signal and, in particular, an improved signal-to-noise ratio are produced.

### BRIEF DESCRIPTION OF THE DRAWINGS

The above-described properties, features and advantages of this invention and the manner in which they are achieved will become clearer and more easily understandable in conjunction with the following description of the exemplary embodiments, which are explained in more detail in conjunction with the drawings. Here, respectively in a schematic illustration:

FIG. 1 shows a cross section through an optical heart rate sensor;

FIG. 2 shows a plan view of a round optical heart rate sensor;

FIG. 3 shows a plan view of an elongate optical heart rate sensor;

FIG. 4 shows a cross section through an optical heart rate sensor that is been placed onto the skin;

FIG. 5 shows a cross section through a further exemplary embodiment of an optical heart rate sensor;

FIG. 6 shows a cross section through a further exemplary embodiment of an optical heart rate sensor;

FIG. 7 shows a cross section through a further exemplary embodiment of an optical heart rate sensor; and

FIG. 8 shows a cross section through a further exemplary embodiment of an optical heart rate sensor.

### DETAILED DESCRIPTION OF ILLUSTRATIVE EMBODIMENTS

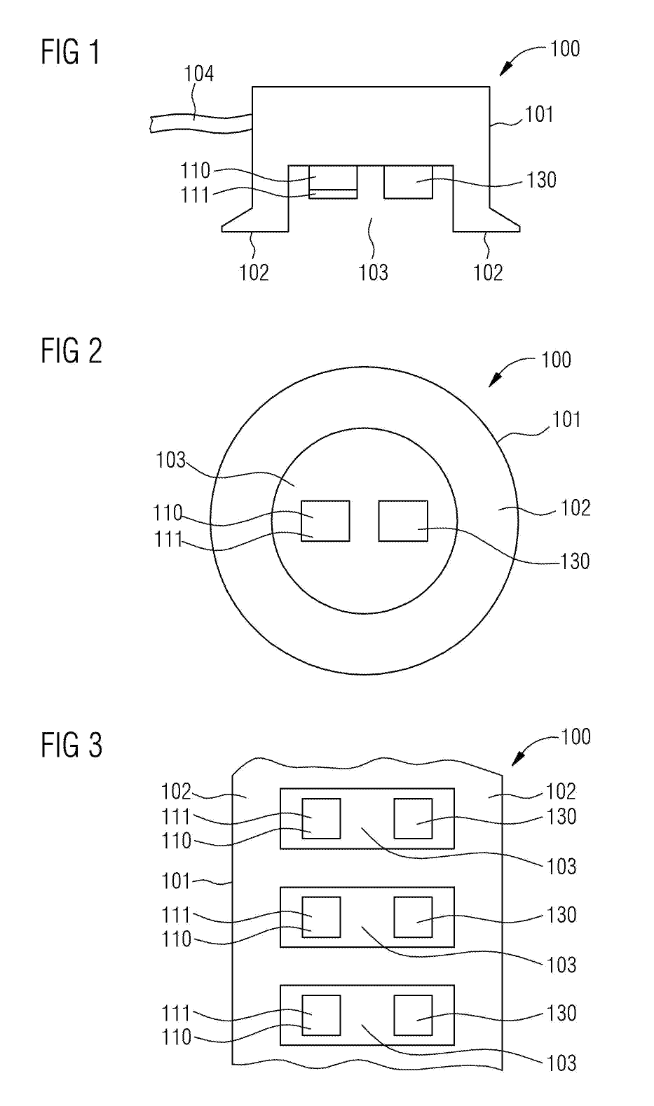

FIG. 1 shows a cross section through an optical heart rate sensor 100. This optical heart rate sensor comprises or consists of a housing 101 with contact faces 102. The contact faces 102 are provided for placing the optical heart rate sensor 100 onto the skin. Moreover, the housing 101 comprises a recess 103 and a data connection 104. In the exemplary embodiment of FIG. 1, the data connection 104 is realized by a cable. However, it is likewise conceivable for the data connection 104 to be realized by means of a radio module. A blue light-emitting diode no with a conversion phosphor 111 is situated within the recess 103 of the housing 101. Moreover, a photodetector 130 is situated in the recess 103. The blue light from the light-emitting diode no is converted into green-yellow light in the conversion phosphor in. The photodetector 130 is configured to detect variations in the light intensity.

FIG. 2 shows a plan view of a round, optical heart rate sensor 100. Here, the viewing direction has been selected in such a way that the contact face 102 of the housing 101 and the recess 103 of the housing 101 are visible. The blue light-emitting diode 110, which is covered by the conversion phosphor in, is situated within the recess 103. The conversion phosphor 111 converts the blue light from the light-emitting diode no into green-yellow light. Moreover, the photodetector 130 is situated in the recess 103. A plurality of light-emitting diodes no with a conversion phosphor in and a plurality of photodetectors 130 may also be provided within the recess 103.

FIG. 3 shows a plan view of an elongate optical heart rate sensor 100. It consists of a housing 101 with a contact face 102 and a plurality of recesses 103, with the recesses 103 being rectangular. A blue light-emitting diode no with a conversion phosphor 111 is situated in each recess 103. Likewise, a photodetector 130 is situated in each recess 103. Provision can also be made for more than one light-emitting diode no with a conversion phosphor in and/or more than one photodetector 130 to be attached in a recess 103. The optical heart rate sensor 100 of this exemplary embodiment may also be embodied as an armband wherein, in that case, the sensor then may be guided, for example, completely around the wrist.

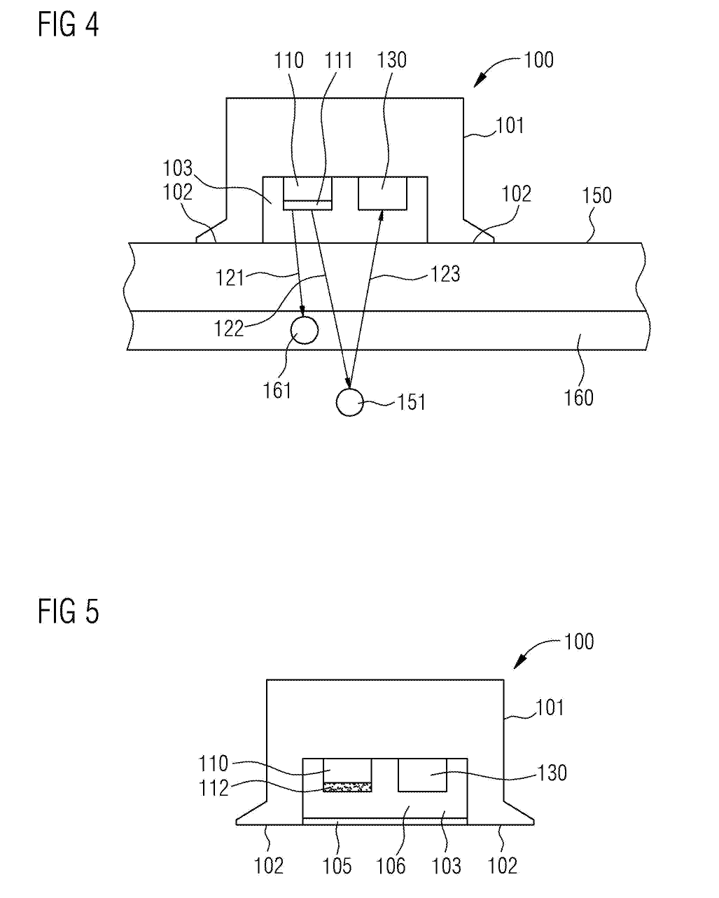

FIG. 4 shows the functionality of the optical heart rate sensor wo from FIG. 1. With its contact faces 102, the optical heart rate sensor 100 lies on the skin. A first light ray 121 is emitted by the blue light-emitting diode 110, converted into green-yellow light in the conversion layer in, and then impinges on a hemoglobin molecule 161 within an artery 160. The first light ray 121 is absorbed by the hemoglobin molecule 161. A second light ray 122 is likewise emitted by the blue light-emitting diode no and converted into green-yellow light in the conversion layer 111. This second light ray 122 passes through the artery 160 and impinges on a tissue particle 151. The second light ray 122 is scattered at the tissue particle 151. The scattered light ray 123 impinges on the photodetector 130. The pulse or the heartbeat changes the number of hemoglobin molecules 161 in the artery 160. As a result, there is a change in the portion of light rays that are absorbed by the hemoglobin molecules 161 when compared to the number of light rays that are scattered by tissue particles 151. As a result of this change in the ratio, there is also a change in the intensity of the light detected in the photodetector 130. This change in intensity is proportional to the heart rate or the pulse. As a result, the heart rate or the pulse can be deduced from the change in the intensity in the photodetector 130.

In an exemplary embodiment, the green-yellow light, which arises from the conversion of the blue light from the light-emitting diode no in the conversion phosphor 111, has a portion of at least 25% in the wavelength range between 540 and 585 nm, while at most 25% of the light has a wavelength longer than 600 nm.

In an exemplary embodiment, the blue light-emitting diode no is an InGaN LED.

In an exemplary embodiment, the blue light-emitting diode no has a wavelength with a maximum intensity that lies between 400 nm and 450 nm.

In an exemplary embodiment, the InGaN LED has an overall efficiency of at least 40%. This means that at least 40% of the energy applied for the light-emitting diode is converted into blue light of the light-emitting diode.

In an exemplary embodiment, the conversion phosphor 111 comprises cerium-doped lutetium aluminum garnet.

In an exemplary embodiment, the cerium concentration in the lutetium aluminum garnet is 1%.

FIG. 5 shows a cross section through a further exemplary embodiment of the optical heart rate sensor 100. The design substantially corresponds to that of the optical heart rate sensor from FIG. 1. In this case, cerium-doped lutetium aluminum garnet, which is cast in powder form into an epoxy resin 112, is provided as a conversion phosphor. Here, the powder of the cerium-doped lutetium aluminum garnet is indicated in FIG. 5 by dots within the epoxy resin 112. The grain size of the cerium-doped lutetium aluminum garnet powder is several micrometers in this case. Moreover, the optical heart rate sensor 100 in FIG. 5 has a cover 105. This cover 105 seals the recess 103 such that the blue light-emitting diode no and the conversion phosphor, which is situated in the epoxy resin 112, and also the photodetector 130 are protected from environmental influences. Here, the cover 105 consists of a material that is transmissive for the green-yellow light that arises in the conversion phosphor. Provision can additionally be made for the recess 103 to be filled with the material 106 that is likewise transmissive for the green-yellow light.

In an exemplary embodiment, the material that is transmissive for the green-yellow light is a silicone, a plastic or a ceramic.

In an exemplary embodiment, the conversion phosphor 111 comprises quantum dots with a diameter of between 2 and 6 nm.

In an exemplary embodiment, the quantum dots comprise mercury sulfide, lead sulfide, cadmium sulfide, cadmium selenide, indium arsenide or indium phosphide.

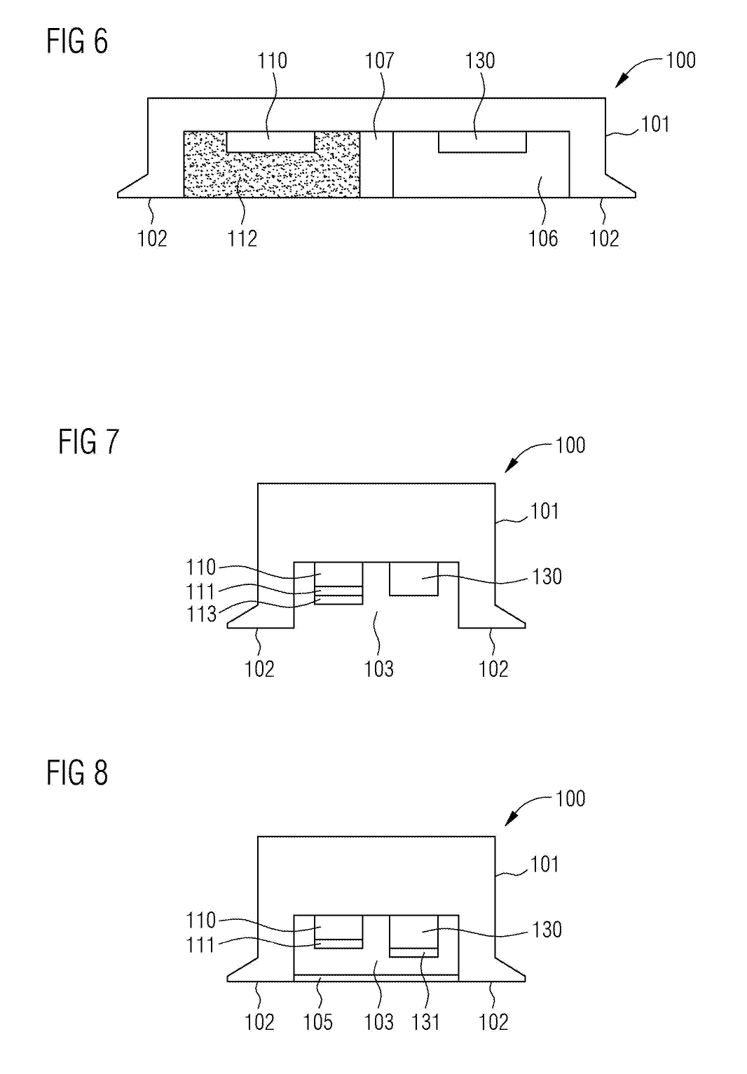

FIG. 6 shows a further cross section through an optical heart rate sensor 100, which consists of a housing 101 with a blue light-emitting diode no and a photodetector 13o. The housing 101 comprises a contact face 102 and a recess, with the blue light-emitting diode no and the photodetector 130 being arranged within the recess. A partition wall 107 that is opaque to light is arranged between the blue light-emitting diode no and the photodetector 13o, as a result of which the recess is subdivided into two regions. In the region of the blue light-emitting diode 110, the recess is filled with an epoxy resin containing a powder made of cerium-doped lutetium aluminum garnet. In the region of the photodetector 130, the recess is filled with a material 106 that is transmissive for the green-yellow light. As a result of this design, it is possible to realize particularly flat optical heart rate sensors.

FIG. 7 shows a further cross section through an optical heart rate sensor 100 which substantially corresponds to the optical heart rate sensor in FIG. 1. In addition to the conversion phosphor iii, the blue light-emitting diode no comprises a filter 113. This filter is configured in such a way that it is transmissive for the green-yellow light in a wavelength range between 540 nm and 585 nm. As a result of this, it is possible, for example, to ensure that no blue light that was not converted in the conversion layer 111 leaves the light source. This minimizes scattered light, as a result of which an improved determination of the heart rate becomes possible.

FIG. 8 shows a further exemplary embodiment of an optical heart rate sensor 100, wherein the optical heart rate sensor 100 substantially corresponds to the optical heart rate sensor in FIG. 1. The photodetector 130 comprises a filter 131, which is transmissive for green-yellow light in the wavelength range between 540 nm and 595 nm. In this exemplary embodiment, scattered light, which is unwanted for the detection of the heart rate, is filtered out in front of the photodetector. In this case too, the signal-to-noise ratio can be improved as a result thereof. The optical heart rate sensor 100 likewise comprises a cover 105 in this exemplary embodiment, said cover 105 being intended to protect both the light source and the photodetector from environmental influences.

In a further exemplary embodiment, the filter, which is transmissive for the green-yellow light in a wavelength range of between 540 nm and 585 nm, is integrated into the cover 105.

Even though the invention was described and illustrated more closely in detail by the preferred exemplary embodiment, the invention is not restricted by the disclosed examples and other variations may be derived therefrom by a person skilled in the art without departing from the scope of protection of the invention.

1-12. (canceled)

13. An optical heart rate sensor comprising:
at least one light source; and
at least one photodetector,
wherein the light source comprises a blue light-emitting diode with a conversion phosphor, and
wherein the conversion phosphor converts the blue light into a green-yellow light.

14. The optical heart rate sensor according to claim 13, wherein at least 25% of the converted light has a wavelength of between 540 nm and 585 nm and at most 25% of the converted light has a wavelength that is longer than 600 nm.

15. The optical heart rate sensor according to claim 13, wherein the blue light-emitting diode is an InGaN LED.

16. The optical heart rate sensor according to claim 15, wherein the InGaN LED has an overall efficiency of at least 40%.

17. The optical heart rate sensor according to claim 13, wherein the blue light-emitting diode has a wavelength with a maximum intensity that lies between 400 nm and 470 nm.

18. The optical heart rate sensor according to claim 13, wherein the conversion phosphor comprises cerium-doped lutetium aluminum garnet.

19. The optical heart rate sensor according to claim 18, wherein the conversion phosphor is a powder in another material, and wherein the other material may be an epoxy resin, a silicone, a plastic or a ceramic.

20. The optical heart rate sensor according to claim 18, wherein a cerium concentration in the lutetium aluminum garnet is 1%.

21. The optical heart rate sensor according to claim 20, wherein the conversion phosphor is a powder in another material, and wherein the other material may be an epoxy resin, a silicone, a plastic or a ceramic.

22. The optical heart rate sensor according to claim 13, wherein the conversion phosphor comprises quantum dots with a diameter of between 2 nm and 18 nm.

23. The optical heart rate sensor according to claim 22, wherein the quantum dots comprise mercury sulfide, lead sulfide, cadmium sulfide, cadmium selenide, indium arsenide or indium phosphide.

24. The optical heart rate sensor according to claim 13, wherein the light source or the photodetector comprises a filter which is transmissive for some of the converted light.

25. The optical heart rate sensor according to claim 24, wherein the filter is transmissive in a wavelength range from 540 nm to 585 nm.
